(19) United States
(12) Patent Application Publication
(10) Pub. No.: US 2012/0010650 A1
(43) Pub. Date: Jan. 12, 2012

(54) Universal Capture Assembly

(76) Inventor: Thomas SOS, New York City, NY (US)

(21) Appl. No.: 13/238,149

(22) Filed: Sep. 21, 2011

Related U.S. Application Data
(62) Division of application No. 12/333,815, filed on Dec. 12, 2008.
(60) Provisional application No. 61/013,946, filed on Dec. 14, 2007.

(51) Int. Cl. A61F 2/01

(52) U.S. Cl. 606/200

(57) ABSTRACT

The present invention relates to a universal capture assembly for use with an implantable device such as a filter and method of use. The universal capture assembly includes a body with a neck having a reduced cross-sectional diameter with respect to the body and a larger diameter neck extending therefrom. The reduced diameter neck and larger diameter neck are separated by a disk shaped collar positioned there between. The body further includes a plurality of grooves formed in an outer surface (longitudinal relative to the longitudinal axis of filter) thereof wherein the plurality of grooves form a plurality of projections that engage and retain the loop of a snare, or other similar retrieval device, with ease and accuracy. The body is designed to include at least two or more, longitudinal grooves and projections. The universal capture is attached to the implantable device by the larger diameter neck portion. The universal capture assembly can include a dual capture assembly wherein the assembly is positioned at opposing ends of the implanted device such that access can be obtained either by femoral, jugular, venous, arterial or other routes.

### CROSS-REFERENCE TO RELATED APPLICATIONS

This application claims priority to pending U.S. provisional patent application Ser. No. 61/013,946 filed on Dec. 14, 2007, all of which is incorporated herein by reference in its entirety.

### FIELD OF THE INVENTION

The present invention relates to an assembly and method that aids in retrieving an implanted device. More particularly, the present invention relates to a universal capture assembly for use with an implantable device, such as a filter device, and a method of using the same to retrieve the implanted device.

### BACKGROUND OF THE INVENTION

Devices, such as vena cava filters, are used to capture potentially fatal blood clots at an anatomical location where they may pose less risk of pulmonary embolism for the patient. Since the vast majority of blood clots originate from the lower body, filters are mainly placed in the inferior vena cava. The filter includes a filtering portion designed to capture blood clots and an anchoring portion designed to anchor the filter at a desired location in the vessel and prevent migration of the device. Some filters are designed to be retrievable thereby allowing the physician to remove the implanted filter after it is no longer needed. Typically, retrievable filters include a hook at one end of the filter for engagement with a snare loop or other retrieval element for removal.

FIG. 1 depicts a prior art filtering device 1 having legs 3 and a retrieval hook 2. The retrieval hook 2 allows for retrieval of the filter device 1 from a vessel 61 using a snare sheath device 63 or other retrieval device known in the art. Prior art filters 1 are difficult to remove because it is hard to properly align the loop 64 of the snare sheath 63 with the hook 2. Further, the radial orientation of the hook opening 4 within the vessel is not visible under two-dimensional images generated by fluoroscopy or ultrasound. As a result, it is often difficult for the physician to engage the filter and position the snare loop 64 within the opening 4 of the hook 2. Misaligned or tilted filters and variants in anatomy of the vessel also contribute to difficulty in properly aligning the snare loop 64 within the opening 4 in the hook 2. Accordingly, it is desirable to provide a retrieval capture assembly that allows for an ease of positioning of a retrieval device, such as the snare, about the retrieval capture assembly at any given angle or radial orientation so as to minimize the likelihood of having to re-position the snare in order to successfully engage, retain and remove the filter.

### BRIEF SUMMARY OF THE DISCLOSURE

A universal capture assembly for use with a retrievable device, such as a blood clot filter, wherein one embodiment of the universal capture assembly includes a mushroom-shaped body with a neck having a reduced cross-sectional diameter with respect to and extending from the body. The mushroom-shaped body further includes a plurality of grooves formed in a surface (longitudinal relative to the longitudinal axis of filter) thereof that creates a plurality of projections designed to engage and retain the snare and its sheath, or other similar retrieval device, with ease and accuracy. The mushroom-shaped body is designed to include at least two or more, longitudinal grooves and projections.

In another aspect of the invention, a universal capture assembly for use with a retrievable device, such as a blood clot filter, wherein the universal capture assembly includes a ball-shaped body with a neck having a reduced cross-sectional diameter with respect to and extending from the body. The ball-shaped body further includes a plurality of grooves formed in a surface (longitudinal relative to the longitudinal axis of filter) thereof that create a plurality of projections designed to engage and retain the snare and its sheath, or other similar retrieval device, with ease and accuracy. The ball-shaped body is designed to include two or more longitudinal grooves and projections.

In another aspect of the invention, a universal capture assembly for use with a retrievable device, such as a blood clot filter, wherein the universal capture assembly includes a triangular or pyramid-shaped body with a neck having a reduced cross-sectional diameter with respect to and extending from the body. The triangular or pyramid-shaped body further includes a plurality of grooves formed in a surface (longitudinal relative to the longitudinal axis of filter) thereof that create a plurality of projections designed to engage and retain the snare and its sheath, or other similar retrieval device, with ease and accuracy. The triangular or pyramid-shaped body is designed to include two or more longitudinal grooves and projections.

In another aspect of the invention, a universal capture assembly for use with a retrievable device, such as a blood clot filter, wherein the universal capture assembly includes a box-shaped body with a neck having a reduced cross-sectional diameter with respect to and extending from the body. The box-shaped body portion further includes a plurality of grooves formed in a surface (longitudinal relative to the longitudinal axis of filter) thereof that create a plurality of projections designed to engage and retain the snare and its sheath, with ease and accuracy. The box-shaped body is designed to include two or more longitudinal grooves and projections.

In another aspect of the invention, a universal capture assembly for use with a retrievable device, such as a blood clot filter, wherein the universal capture assembly includes a crescent-shaped body with a neck having a reduced cross-sectional diameter with respect to and extending from the body. The crescent-shaped body further includes a plurality of grooves formed in a surface (longitudinal relative to the longitudinal axis of filter) thereof that create a plurality of projections designed to engage and retain the snare and its sheath, with ease and accuracy. The crescent-shaped body is designed to include two or more, preferably four, longitudinal grooves and projections.

In another aspect of the invention, a universal capture assembly for use with a retrievable device, such as a blood clot filter, wherein the universal capture assembly includes a spear-shaped body with a neck having a reduced cross-sectional diameter with respect to and extending from the body. The spear-shaped body includes a plurality of grooves and projections formed in a surface (longitudinal relative to the longitudinal axis of filter) thereof to engage and retain the snare and its sheath, or other similar retrieval device, with ease and accuracy. The spear-shaped body is designed to include three longitudinal grooves and projections.

In another aspect of the invention, a universal capture assembly for use with a retrievable device, such as a blood clot filter, wherein the universal capture assembly includes an hour-glass shaped body with a neck having a reduced cross-sectional diameter with respect to and extending from the body. The hour-glass shaped body portion includes a plurality of grooves and projections formed in a surface (longitudinal relative to the longitudinal axis of filter) thereof to engage and retain the snare and its sheath, or other similar retrieval device, with ease and accuracy. The hour-glass shaped body is designed to include only two longitudinal grooves and projections.

In another aspect of the invention, a dual universal capture assembly for use with a retrievable device, such as a symmetrical filter or stent that includes two capture assemblies attached to the proximal and distal ends of the filter or stent. Each capture assembly includes any one of a mushroom, ball, triangular/pyramidal, box, crescent, spear or hour-glass shaped body, or any combination thereof attached, respectively thereto. The dual universal capture assembly allows for removal of the device from various access approaches, such as a femoral or jugular or brachial approach.

In yet another aspect of the invention, a universal capture assembly for use with a retrievable device, such as an occluding coil that includes a body portion having a proximal and a distal end thereof with a mushroom, ball, triangular/pyramidal, box, crescent, spear or hour-glass shaped body attached to one end. The universal capture assembly allows for the occluding coil to be removed or repositioned after initial deployment.

A further aspect of the invention includes a method of retrieval for an implanted device such as a filter. The filter is retrieved from the walls of the vessel by first inserting a snare device, into the vessel. The snare is then advanced until a loop, formed at an end of the snare, is positioned near to the universal capture assembly. The filter is then seized by looping the snare around the neck of the universal capture assembly. Gentle tension is applied to the proximal end of the loop snare while the loop snare sheath is advanced over the snare to tighten the loop of the snare, and the loop is securely grasped about the projections and retained within a single longitudinal groove of the universal capture assembly. Tension is maintained at the proximal end of the snare while the retrieval sheath is advanced over the distal end of the snare sheath and snare loop. This action encapsulates the universal capture assembly of the filter in the retrieval sheath lumen thereby causing the filter to begin to collapse and the legs to disengage from the vessel wall. Further advancement of the retrieval sheath causes the filter device body to become completely collapsed with the retrieval sheath coaxially surrounding the collapsed filter body. Once the filter is entirely encapsulated within the lumen of the retrieval sheath, the retrieval sheath and filter are then removed from the vessel.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is a plan view of a prior art filter device in an expanded state inside a vessel with a retrieval hook and a snare device just prior to attachment onto the hook of the filter before retrieval.

FIG. 2 is a plan view of an expanded filter with the universal capture assembly of one exemplary embodiment, according to the present invention.

FIG. 3A is an isometric view of the universal capture assembly of the device as shown in FIG. 2, according to the present invention.

FIG. 3B is a plan view of the universal capture assembly of the device as shown in FIG. 2, according to the present invention.

FIG. 3C is a top view of the universal capture assembly of the device as shown in FIG. 2, according to the present invention.

FIG. 4 is a side view of a filter device of the present invention in a deployed state inside a vessel depicting a snare device just prior to attachment onto the universal capture assembly before retrieval.

FIG. 5A is a partial plan view of a method of the present invention detailing the filter device of FIG. 2 in a deployed state with a snare loop positioned around the neck of the universal capture assembly before retrieval.

FIG. 5B is a partial plan view of a method of the present invention detailing the filter device of FIG. 2 in a deployed state with the snare loop tightened around the neck of the universal capture assembly.

FIG. 5C is a partial plan view of a method of the present invention detailing the filter device of FIG. 2 with the retrieval sheath coaxially surrounding the universal capture assembly.

FIG. 5D is a partial plan view of filter with universal capture assembly in an entirely collapsed state but not completely within the retrieval sheath just prior to removal from the vessel.

FIG. 5E is a partial plan view of entire filter with universal capture assembly collapsed and completely within the retrieval sheath just prior to removal from the vessel.

FIG. 6A is cross-sectional view taken along Section A-A in FIG. 5D of the universal capture assembly detailing engagement of a snare about the reduced neck, according to the present invention.

FIG. 6B is cross-sectional view taken along Section A-A in FIG. 5D of the universal capture assembly detailing engagement of a snare within at least two of the longitudinal grooves, according to the present invention.

FIG. 7 illustrates isometric views of alternate embodiments of the universal capture assembly, according to the present invention.

FIG. 8 illustrates isometric views of alternate embodiments of the universal capture assembly, according to the present invention.

FIG. 9 is a plan view of a stent device with a dual universal capture assembly, according to the present invention.

FIG. 10 is a plan view of an occluding coil device with a universal capture assembly, according to the present invention.

FIG. 11 is a plan view of an occluding coil device with a dual universal capture assembly, according to the present invention.

### DETAILED DESCRIPTION OF THE INVENTION

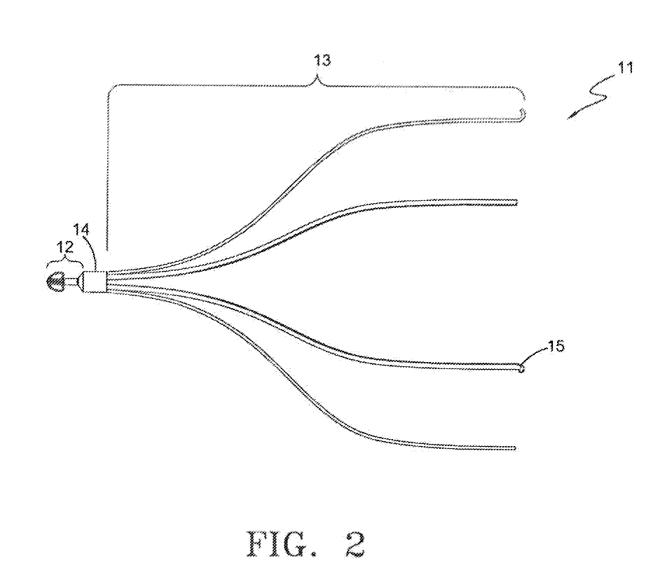

Referring to FIG. 2, there is shown an embodiment of the present invention of a universal capture assembly 12 of an expanded medical device, such as, a vena cava filter 11. When deployed in the path of the bloodstream, typically in the inferior vena cava, the filter 11 captures blood clots of a predetermined size and prevents them from traveling further toward the lungs. The vena cava filter 11 is comprised of legs 13 and a universal capture assembly 12. Legs 13 converge to an end hub 14. The purpose of the legs 13 is to capture blood clots, anchor the filter 11 to the vena cava with distal prongs or barbs 15 to prevent the filter 11 from migrating. The end hub 14 provides for a connection location for the universal capture assembly 12. The universal capture assembly 12 allows retrieval of the filter 11 from the vessel using a snare device or other retrieval device known in the art.

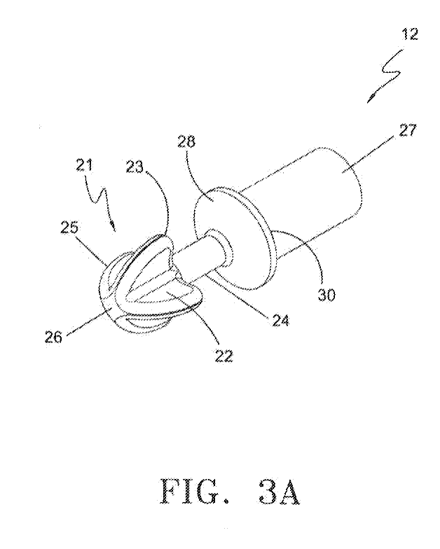

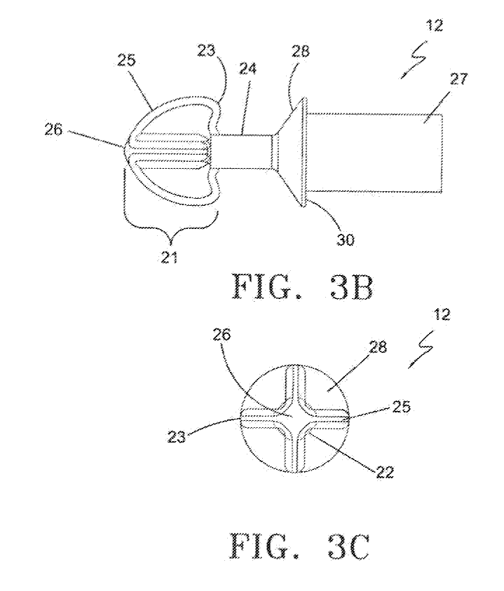

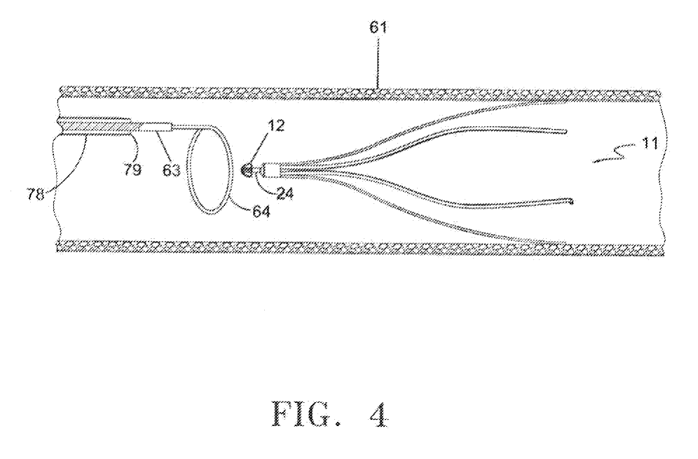

In the preferred embodiment, as shown in FIGS. 3A-3C, the universal capture assembly 12 includes a mushroom-shaped body 21 with a plurality of longitudinal grooves 22 formed therein to create a plurality of projections 23 designed to capture the loop of a snare, or other similar retrieval device, with ease and accuracy. The mushroom-shaped body 21 is designed to include two or more longitudinal grooves 22 and projections 23, preferably four (see FIG. 3). The outer surface 25 of the projections 23 are arcuate or curved and meet at a slightly rounded apex 26. The mushroom-shaped body 21 includes a reduced diameter neck 24 and larger diameter neck 27 extending therefrom. The reduced diameter neck 24 and larger diameter neck 27 are separated by a disk shaped collar 28 positioned there between. As clearly shown in FIGS. 3B and 3C, the disk shaped collar 28 extends beyond the outer circumference of the larger diameter neck 27. This extension provides for a seat 30, shown in FIGS. 3A and 3B. The universal capture assembly 12 is attached to the filter 11 via the larger diameter neck portion 27. The outer-most cross-sectional diameter of body 21 of the universal capture assembly 12 is of the same cross-sectional diameter as the diameter of the disk shaped collar 28 (See FIG. 3C). Thus, the universal capture assembly 12 does not increase the overall cross-sectional profile of the filter 11 which is advantageous in minimizing sheath size and trauma to the patient during deployment and retrieval.

Although various dimensions can be encompassed within the scope of the invention, the preferred general dimensions of the universal capture assembly include a length (excluding the larger diameter neck length) of 0.120 inches that minimizes the overall length of the device, a capture body 21 diameter of 0.072 inches that allows for retrieval using at least a 7F retrieval sheath 78 and a reduced neck width of 0.020 inches that provides for a capture width of 0.026 inches. The preferred capture body 21 width of 0.026 inches is a width sufficient to maintain the structural strength of the reduced neck portion 24; thereby permitting the reduced neck portion 24 to withstand the forces applied thereto during withdrawal or repositioning of the filter 11 without bending, fracture or becoming disengaged from the filter. The universal capture assembly 12 can be formed of medical grade stainless steel, shape memory nitinol, titanium or other materials known in the art. Capture assembly 12 may be formed as a single member along with the filter 11, or the universal capture assembly 12 can be formed separately from the filter 11 and attached thereto. There are various techniques available for attaching the universal capture assembly to the filter. Typically, retrieval elements are attached to the filter by techniques such as laser welding, plasma welding, welding brazing, welding, soldering, pin and hole (or other mechanical attachment and hole), crimping, hub and cylinder, cover and wire attachment or bonding. The universal capture assembly 12, of the present invention, is not limited to any one technique for attachment to the filter 11. In some instances, the larger diameter neck 27 is the point of reference for attaching the universal capture assembly 12 to the filter 11. In other instances, the seat portion 30 aids as a positioning mechanism that rests flush and comfortably against the end hub 14 of the filter 11. Other designs may be used to connect the universal capture assembly 12 to other implantable device designs such as a stent or occluding coil. The attachment technique used is governed solely by the type of device and the physical design of the device.

Further, the universal capture assembly 12 has increased strength relative to traditional hooks 2 because the mass of the assembly allows for greater tensile pulls without bending or otherwise distorting the assembly. For instance, wire hooks are easily bent particularly if there is a lot of force required for retrieval due to endothelial overgrowth. However, the universal capture assembly 12 allows for the force applied thereto to be distributed equally about the projections, thereby eliminating an excessive amount of force being applied at one concentrated location. Having the applied forces distributed decreases the likelihood that the universal capture will become bent, fractured or disengaged from the filter during the retrieval process.

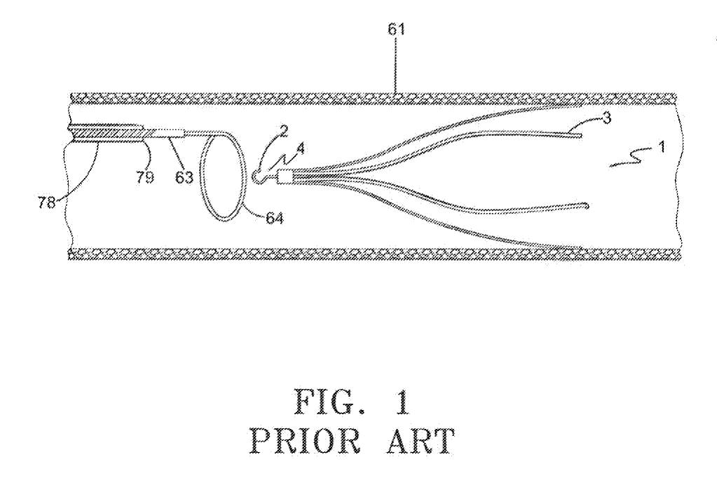

The method of retrieving prior art filter 1 is shown in FIG. 1, wherein the retrieval sheath 78 which coaxially surrounds snare sheath 63, is inserted into the vessel 61 and advanced to the filter 1. The snare sheath 63 is then further advanced beyond the distal end 79 of the retrieval sheath 78. The retrieval hook 2 of the filter 1 is captured by looping the snare wire 64 of the snare sheath 63 around hook 2 and applying tension to engage the hook 2. As illustrated by the position of the hook opening 4 relative to the snare loop 64 in FIG. 1, when snare sheath 63 is advanced to pull the snare loop 64 taut around the hook 2, the loop 64 may not engage the hook 2 or the loop 64 may become engaged around the hook 2, but not be positioned within the hook opening 4. As a result, the loop 64 may fail to engage the hook 2, requiring additional maneuvering of the snare loop 64 in an attempt to properly align the snare loop 64 relative to the hook opening for engaging the hook 2.

Standard imaging techniques, such as fluoroscopy or ultrasound, which both produce two-dimensional images, are unable to accurately identify the angle or specific radial orientation of the hook 2 within the vessel 61. Without a three-dimensional visual image of the filter 1, the physician must “blindly” maneuver the snare loop 64 in the general area of the hook in an attempt to capture the hook opening 4. In addition, some imaging modalities, such as ultrasound, may not have the resolution clarity to allow the physician to identify the hook 2 at all.

Another problem related to prior art hook retrieval designs involves filters 1 that have either been deployed at a misaligned angle or have become tilted within the vessel 61 over the period of implantation. When this occurs, the retrieval hook 2 is no longer centered within the vessel lumen and may be so misaligned that the retrieval hook 2 rests up against the vessel wall 61. Retrieval of a device that has the retrieval hook 2 in contact with the vessel wall 61 may be very difficult, particularly if the hook opening 4 is abutting the vessel wall 61.

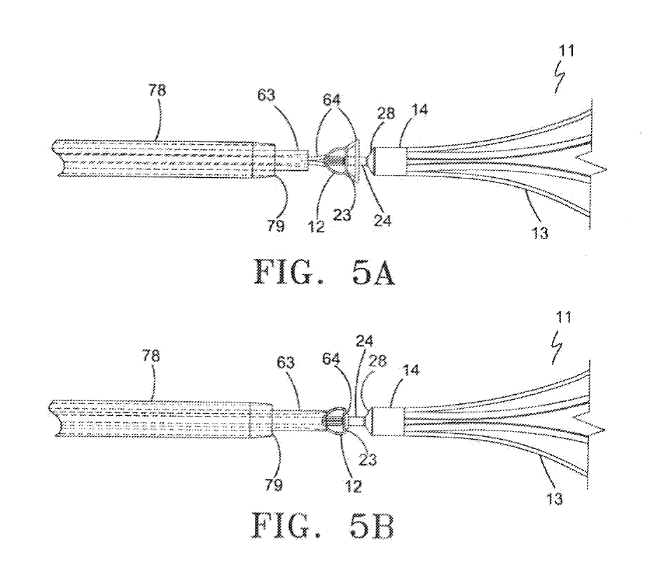

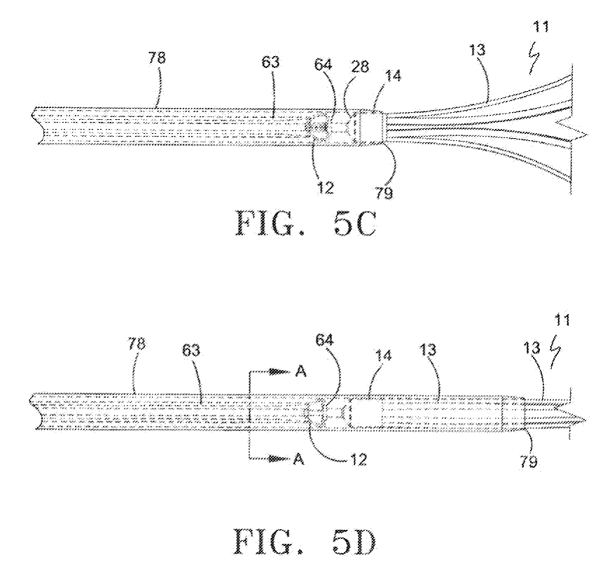

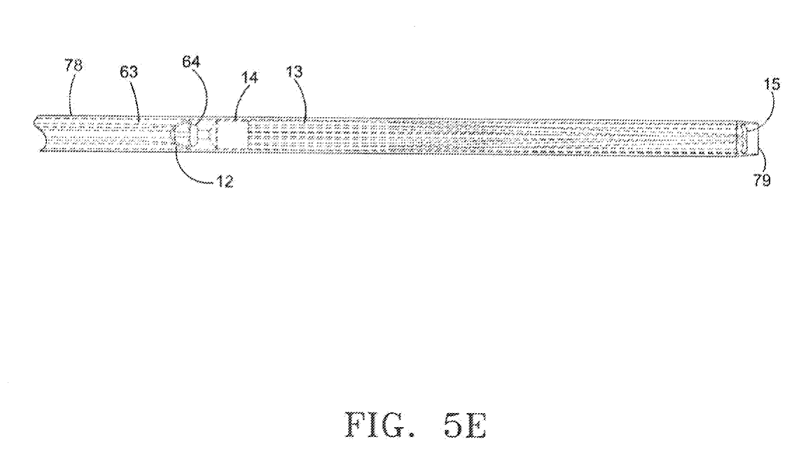

In contrast, FIG. 4 illustrates the ease of positioning of the snare device with respect to the universal capture assembly 12 of the current invention. Wire loop 64 of the snare sheath 63, when placed, is easily and completely accessible for retention by projections 23 and longitudinal grooves 22 (see FIG. 3A) of the universal capture assembly 12 from multiple or various locations thereabout without having to maneuver or manipulate the snare wire loop 64 into a specific orientation or position relative to the projections 23. As shown, positioning of the snare wire loop 64 of the snare sheath 63 with respect to the universal capture assembly 12 is not affected by the specific radial orientation of the hook 2, as with the prior'art of FIG. 1. Instead, the physician simply engages the snare wire loop 64 around the reduced diameter neck 24 and advances the snare sheath 63 forward toward filter 11 (see FIGS. 5A and 5B). Advancement of the snare sheath 63 will progressively decrease the overall diameter of snare wire loop 64 resulting in the loop tightening around the reduced diameter neck region 24. The snare wire loop 64 will be automatically directed into one of the grooves 22 as additional forward tension is applied to tighten the loop 64.

Referring to FIGS. 5A-5E, additional details of the method of the current invention are further illustrated. FIGS. 5A-5E depict enlarged partial plan views of the snare wire loop 64 engaging the universal capture assembly 12 of the filter 11 and the resulting collapse of the filter as it is coaxially encapsulated within the retrieval sheath 78 lumen. Retrieval of the filter 11 is accomplished by inserting a snare sheath 63, with a coaxially surrounding retrieval sheath 78, into the vessel (not shown) and positioning the retrieval device proximate to filter 11. The snare sheath 63 is then advanced beyond the distal end 79 of the retrieval sheath 78, as shown in FIG. 5A. The universal capture assembly 12 is captured by positioning the snare wire loop 64 in the vicinity of the reduced neck 24 of the universal capture assembly 12 between collar 28 and projections 23.

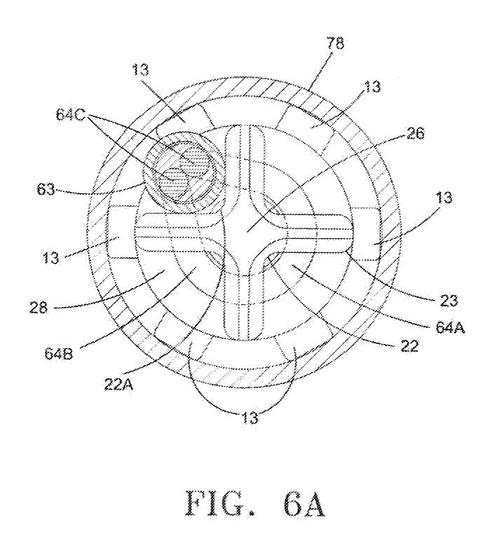

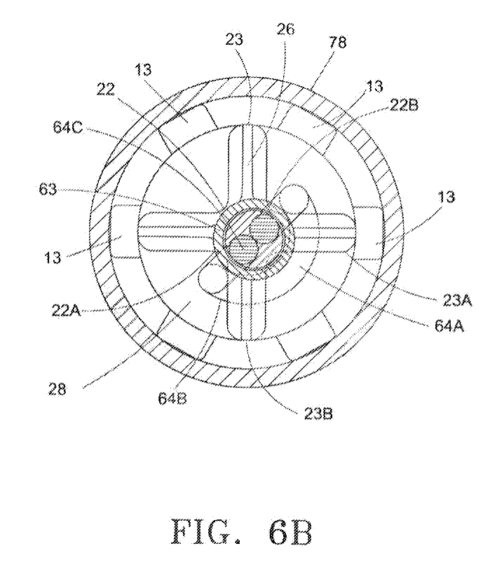

Once the snare wire loop 64 is positioned loosely around neck 24, the snare wire loop 64 is tightened, as illustrated in FIG. 5B. Forward advancement of the snare sheath 63; causes the diameter of the snare wire loop 64 to become smaller as it tightens around the reduced neck 24. The snare wire loop 64 becomes securely engaged against the proximal most edge of neck 24 with the projections 23 acting as capture points for the snare loop 64. Both arms of the snare wire loop 64 encapsulated in the lumen of snare sheath 63 become nestled within a single longitudinal groove 22, as will be described in more detail with reference to FIG. 6A below.

After the loop 64 of the snare sheath 63 is secured tautly about the universal capture assembly 12 by advancing the snare sheath 63 to the filter, tension is maintained at the proximal end of the snare sheath 63, while the retrieval sheath 78 is advanced over the distal end of snare sheath 63. As the retrieval sheath 78 is advanced over the snare sheath 63 distal end, sheath 78 encapsulates the universal capture assembly 12 of the filter 11 within the lumen of the retrieval sheath 78. Referring to FIG. 5C, the universal capture assembly 12 and the end hub 14 of the filter 11 have been enclosed within the forward advancing retrieval sheath 78 lumen.

Further advancement of the retrieval sheath 78, as shown in FIG. 5D, causes the outwardly positioned filter legs 13 to become drawn in toward the center of the vessel lumen. Continued advancement causes the legs 13′ of the filter 11 to disengage from the vessel wall. The filter legs 13 collapse inwardly as the filter 11 becomes encapsulated within the retrieval sheath 78 lumen. Referring to FIG. 5E, once the filter, including the end hub 14, legs 13 and prongs or barbs 15, is completely contained within the retrieval sheath 78, the retrieval sheath 78, snare sheath 63 and filter 11 may be removed from the vessel as a single unit.

FIGS. 6A and 6B illustrate cross-sectional views of the universal capture assembly 12 and filter 11 within the retrieval sheath 78 taken along Section A-A of FIG. 5D. The proximal end apex 26 of the universal capture assembly 12 and the snare retrieval sheath 63 with the wire snare loop 64C within the snare retrieval sheath 63 are depicted with them both coaxially surrounded by the retrieval sheath 78. Filter legs 13 are in a compressed position within the retrieval sheath 78. Multiple projections 23 formed by grooves 22 provide a surface profile that engages and retains the snare wire loop 64(A-C), making it virtually impossible to misalign the snare wire loop 64 relative to the universal capture assembly 12. Grooves 22 further minimize the combined cross-sectional profile of the snare 63 and filter 11. When snare wire loop 64 is taut, it becomes positioned against the reduced diameter neck 24 between collar 28 and the distal-facing surface projections 23. The embodiment in FIG. 6A depicts end portion 64A of snare wire loop 64 positioned distally of projections 23 abutting grooves 22. Snare wire portions 64B are also positioned distally of projections 23 abutting grooves 22. Both snare wire portions 64C are positioned within a single groove 22A before entering the snare sheath 63, which is also positioned within groove 22A.

Alternatively, snare wire loop 64 may be captured and retained by two projections 23 as shown in FIG. 6B. The end portion 64A of snare wire loop 64 is positioned between collar 28 and the distal facing surface of two projections, 23A and 23B with snare wire 64B being firmly positioned and seated within grooves 22A and 22B. The wire portions 64C then enter the snare sheath 63 lumen, which is also positioned within the same groove 23. In another aspect of the invention, the snare wire loop 64 may be captured and retained by a single projection or by a subset of the total number of projections 23 of capture assembly 12. Thus, the user may successfully capture and retrieve a device by capturing only a portion of the total number of capture assembly projections 23.

Additionally, the snare wire loop 64 and snare sheath 63 do not increase the overall cross-sectional diameter of collapsed filter within the sheath, as is clearly shown in FIGS. 6A and 6B. The sheath 63 is positioned within groove 23 and as is positioned totally within the maximum cross-sectional diameter of the collapsed filter 11, which allows for a standard size retrieval sheath to be used for the retrieval procedure. When advancing the retrieval sheath 78 over the snare sheath 63 and captured filter 11, the distal end of 79 of retrieval sheath 78 will not get caught or snagged on the snare sheath 63 because it is within the cross-sectional diameter of the collapsed filter. The smooth, arced profile of the universal capture assembly 12 also minimizes the possibility of the retrieval sheath 78 snagging on or otherwise becoming caught during the retrieval procedure.

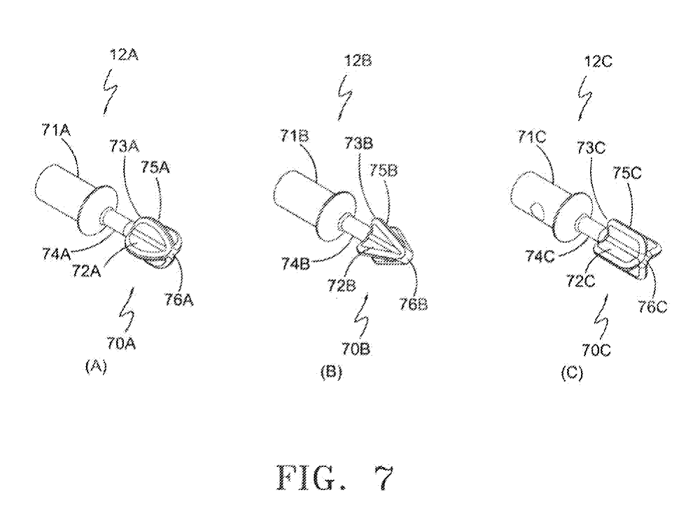

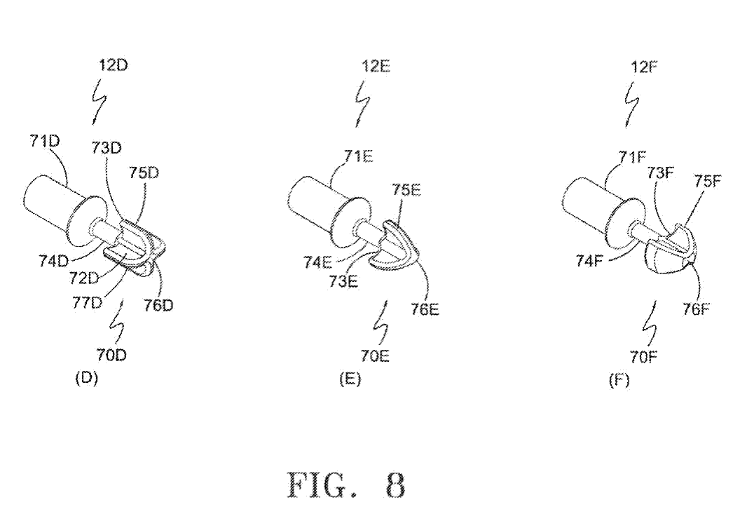

Various embodiments of the universal capture assembly 12 are shown in FIGS. 7 and 8. In one embodiment, as shown in FIG. 7(A), the universal capture assembly 12A includes a ball-shaped body 70A with a plurality of longitudinal grooves 72A formed therein to create a plurality of projections 73A designed to engage and retain the loop of a snare, or other similar retrieval device, with ease and accuracy. The ball-shaped body 70A is designed to include two or more longitudinal grooves 72A and projections 73A. The outer surface 75A of the projections are arcuate or curved. The ball-shaped body 70A includes a reduced diameter neck 74A and a larger diameter neck 71A extending therefrom to ensure connection of the universal capture assembly 12A to a filter or other retrievable device.

In another embodiment, as shown in FIG. 7(B), the universal capture assembly 12B includes a triangular-shaped body 70B with a plurality of longitudinal grooves 72B formed therein to create a plurality of projections 73B designed to capture the loop of a snare, or other similar retrieval device, with ease and accuracy. The triangular-shaped body 70B is designed to include two or more longitudinal grooves 72B and projections 73B. The outer surface 75B of the projections are straight and meet at a slightly pointed apex 76B. The triangular-shaped body 70B includes a reduced diameter neck 74B and a larger diameter neck 71B depending therefrom to ensure connection of the universal capture assembly 12B to a filter or other retrievable device.

In another embodiment, as shown in FIG. 7(C), the universal capture assembly 12C includes a box-shaped body 70C with a plurality of longitudinal grooves 72C formed therein to create a plurality of projections 73C designed to capture the loop of a snare, or other similar retrieval device, with ease and accuracy. The box-shaped body 70C is designed to include four or more longitudinal grooves 72C and hook-shaped form projections 73C. The outer surface 75C of the projections are straight, and the top 76C of the box-shaped body 70C is flat. The box-shaped body 70C includes a reduced diameter neck 74C and a larger diameter neck 71C extending therefrom to ensure connection of the universal capture assembly 12C to a filter or other retrievable device.

In another embodiment, as shown in FIG. 8(D), the universal capture assembly 12D includes a crescent-shaped body 70D with a plurality of longitudinal grooves 72D formed therein to create a plurality of projections 73D designed to capture the loop of a snare, or other similar retrieval device, with ease and accuracy. The crescent-shaped body 70D is designed to include four or more longitudinal grooves 72D and projections 73D. The outer surface 75D of the projections are straight, and the top 76D of the box-shaped body 70D is flat. The straight outer surface 75D is joined to the flat top surface 76D by arcuate or curved elbows 77D. The crescent-shaped body 70D includes a reduced diameter neck 74D and a larger diameter neck 71D extending therefrom to ensure connection of the universal capture assembly 12D to a filter or other retrievable device.

In another embodiment, as shown in FIG. 8(E), there is a universal capture assembly 12E, similar to the preferred embodiment as shown in FIG. 3A, except the universal capture assembly 12E includes a spear-shaped body 70E having only three longitudinal grooves 72E and projections 73E. The outer surface 75E of the projections 73E are arcuate or curved and meet at a slightly rounded apex 76E. Similar, to the previous embodiments, the universal capture assembly 12E includes a reduced diameter neck 74E and larger diameter neck 71E; wherein, the universal capture assembly 12E is attached to a filter or other retrievable device via the larger diameter neck 71E.

In another embodiment, as shown in FIG. 8(F), there is a universal capture assembly 12F, similar to the preferred embodiment as shown in FIG. 3A, except the universal capture assembly 12F includes a hour-glass shaped body portion 70F having only two longitudinal grooves 72F and projections 73F. The outer surface 75F of the projections 73F are arcuate or curved and meet at a slightly rounded apex 76F. Similar, to the previous embodiments, the universal capture assembly 12F includes a reduced diameter neck 74F and larger diameter neck 71F; wherein, the universal capture assembly 12F is attached a filter or other retrievable device via the larger diameter neck 71F.

In all embodiments shown in FIGS. 7 and 8, the surface profiles of the universal capture assemblies may be radiused to form a smooth profile without sharp edges that may damage the vessel, retrieval devices or other interventional devices. As an example, apex 76 and the plurality of projections 73 may radiused to provide an atraumatic surface profile.

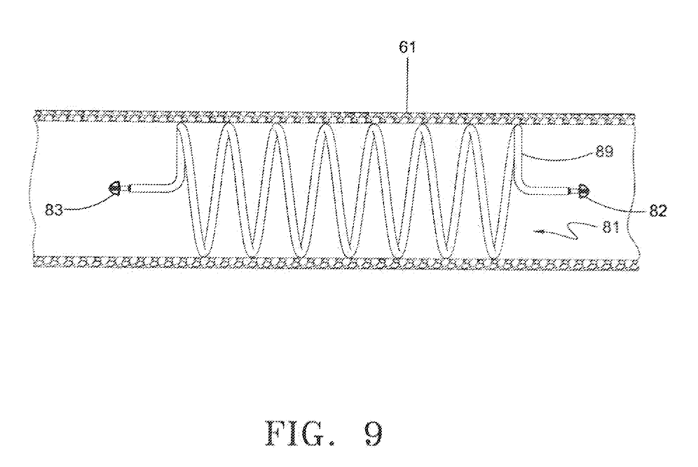

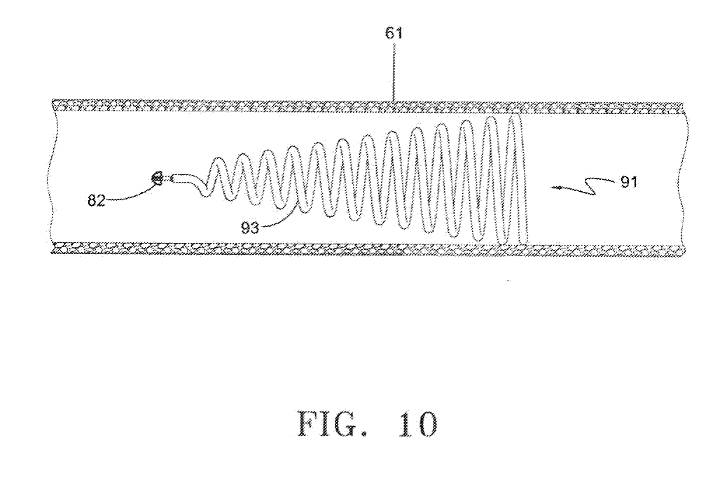

The universal capture assembly 12, of the present invention, may also be used for symmetrically-shaped implanted devices such as filters or stents, which allow the implanted device to be removed from different access approaches such as through the femoral or jugular veins. Arterial approach routes are also within the scope of this invention, such as for stent or embolization coil retrieval. As shown in FIG. 9, a dual universal capture assembly includes capture assembly 82 and 83 which are attached to opposite ends of the stent device 81. Capture assemblies 82 and 83 may be designed with a mushroom 12, ball 12A, triangular/pyramidal 12B, box 12C, crescent 12D, spear 12E or hour-glass 12F shaped body portion attached, respectively thereto. The dual universal capture assemblies 82 and 83 allow for removal of the device 81 from either direction within the vessel or anatomical duct. For example, device 81 may be retrieved by capturing either universal capture assembly 82 with a snare or similar device or may be retrieved by capturing assembly 83. In the case of a vena cava filter, a femoral or jugular approach may be used. The larger diameter neck portions 27, 77A, 77B, 77C, 77D and 77E of the mushroom 12, ball 12A, triangular/pyramidal 12B, box 12C, crescent 12D, spear 12E and hour-glass 12F shaped universal capture assemblies are adapted for attachment; respectively, to the body portion 89 of the retrievable device 81. Having the ability to access and retrieve the device 81 in a venous situation from a femoral or jugular approach, or in an arterial situation, from a femoral or brachial approach, is beneficial in various clinical conditions, including but not limited to, an inability to retrieve through a particular access approach due to the patient's anatomy, disease state or trauma. The dual universal capture assemblies 82 and 83 provide for ease of retrieval from various access approaches.

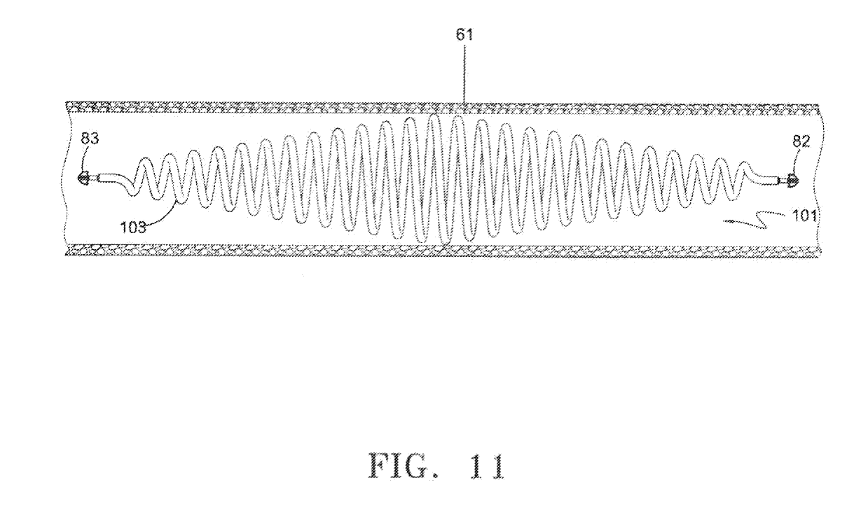

FIG. 10 details the universal capture assembly 82, of the present invention, used with another retrievable device 91, such as an occluding coil, that may include a body portion 93 having an outwardly tapering profile, or an otherwise shaped profile. Universal capture assembly 82 may include a mushroom 12, ball 12A, triangular/pyramidal 128, box 12C, crescent 12D, spear 12E or hour-glass 12F shaped body portion attached to one end of the device 91. The larger diameter neck portions 27, 77A, 77B, 77C, 77D and 12E of the mushroom 12, ball 12A, triangular/pyramidal 12B, box 12C, crescent 12D, spear 12E an hour-glass 12F shaped universal capture assemblies are adapted for attachment; respectively, to the retrievable device 91. Device 91 may be deployed and then recaptured during the same interventional procedure using the universal capture assembly 82 to reposition and redeploy the device within the vessel 61. As shown in FIG. 11, occluding coil device 101 may also include a dual universal capture assembly 82 and 83 attached to opposite ends of the coil device body 103 to reposition or redeploy the device 101 from either direction within the vessel or anatomical duct.

It should be noted, that the universal capture assembly of the present invention may be used for any retrieval device including stents, temporary embolic filters, occlusion devices, and other medical devices. An unlimited number of configurations for the present invention could be realized. The foregoing discussion describes merely exemplary embodiments illustrating the principles of the present invention, the scope of which is recited in the following claims. Those skilled in the art will readily recognize from the description, the claims, and drawings that numerous changes and modifications can be made without departing from the spirit and scope of the invention. Accordingly, the scope of the invention is not limited to the foregoing specification.

What is claimed is:

1. A method of retrieving a medical device using a capture assembly including the steps of:
(a) inserting a retrieval tool, including a snare element, into an anatomically tubular structure;
(b) engaging the snare element with the capture assembly;
(c) securing the snare element against a neck or a projection of the capture assembly;
(d) positioning a portion of the snare element within at least one of a plurality of grooves formed in an outer surface of a body of the capture assembly; and
(e) withdrawing the medical device.

2. The method according to claim 1, wherein the capture assembly is selected from the group consisting of mushroom, ball, triangular, box, crescent, spear and hour-glass shaped.

3. The method according to claim 1, wherein the anatomically tubular structure is a vessel.

4. The method according to claim 1, wherein the medical device is selected from the group consisting of a filter, stent, and occluding device.

5. The medical device according to claim 1, wherein the medical device includes two opposing ends.

6. The medical device according to claim 5, wherein said capture assembly includes at least two capture assemblies, each positioned at said opposing ends of the medical device.

7. The medical device according to claim 6 wherein the medical device is selected from the group consisting of a filter, stent, and occluding device.

8. The method according to claim 7, wherein the medical device is retrieved by a venous approach.

9. The method according to claim 8, wherein the medical device is retrieved by either a femoral or jugular approach.

10. The method according to claim 7, wherein the medical device is retrieved by an arterial approach.

11. The method according to claim 10, wherein the medical device is retrieved by either a femoral or brachial approach.

12. A method of repositioning a medical device using a capture assembly including the steps of:
(a) deploying a medical device within an anatomically tubular structure;
(b) inserting a retrieval tool, including a snare element, into the anatomically tubular structure;
(c) engaging the snare element with the capture assembly;
(d) securing the snare element against a neck or a projection of the capture assembly;
(e) positioning a portion of the snare element within at least one of a plurality of grooves formed in an outer surface of a body of the capture assembly; and
(f) withdrawing the medical device.

13. The method according to claim 12, wherein the steps further include the steps of:
(g) redeploying the medical device within the anatomically tubular structure; and
(h) repeating steps (a)-(g) until the medical device is properly positioned.

14. The method according to claim 12, wherein the capture assembly is selected from the group consisting of mushroom, ball, triangular, box, crescent, spear and hour-glass shaped.

15. The method according to claim 12, wherein the anatomically tubular structure is a vessel.

16. The method according to claim 12, wherein the medical device is selected from the group consisting of a filter, stent, and occluding device.
